(19) United States
(12) Patent Application Publication
(10) Pub. No.: US 2024/0382922 A1
(43) Pub. Date: Nov. 21, 2024

(54) METHOD FOR CLOSED-LOOP CONTROL OF THE TEMPERATURE IN A PROCESS ENGINEERING APPARATUS

(71) Applicant: BASF SE, Ludwigshafen am Rhein (DE)

(72) Inventors: Frank HUETTEN, Ludwigshafen am Rhein (DE); Florian WOLFF, Ludwigshafen am Rhein (DE)

(21) Appl. No.: 18/286,598

(22) PCT Filed: Apr. 5, 2022
(86) PCT No.: PCT/EP2022/058969

§ 371 Date: Oct. 12, 2023

(30) Foreign Application Priority Data
Apr. 15, 2021 (EP) 21168533.4

(51) Int. Cl. B01J 19/00; C07C 45/50

(52) U.S. Cl. CPC B01J 19/0013; C07C 45/50; B01J 2219/00063; B01J 2219/0011; B01J 2219/00164; B01J 2219/002; B01J 2219/0022; B01J 2219/00238

(57) ABSTRACT

The invention relates to a method of closed-loop control of the temperature in a chemical engineering apparatus (101, 201, 301, 401), in which, in a primary circuit (102, 202, 302, 402), a liquid is conveyed out of the apparatus (101, 201, 301, 401), fed at least partly to a heat transferer (103, 203, 303, 403) and recycled at least partly back to the apparatus (101, 201, 301, 401), where the heat transferer (103, 203, 303, 403) is cooled or heated by a heat transfer medium in a secondary circuit (104, 204, 304, 404), comprising the steps of:
providing a target value for the temperature of the liquid in the apparatus (101, 201, 301, 401),
detecting an actual value for the temperature of the liquid in the apparatus (101, 201, 301, 401) and
calculating the temperature difference between the actual value and the target value of the liquid in the apparatus (101, 201, 301, 401).
   According to the invention,
a heat flow taken from or added to the liquid in the primary circuit (102, 202, 302, 402) by the heat transferer (103, 203, 303, 403) is ascertained,
a control signal is calculated on the basis of a defined closed-loop control algorithm, where the closed-loop control algorithm is configured such that the control signal is dependent on the heat flow and the temperature difference between the actual value and the target value of the liquid in the apparatus (101, 201, 301, 401), and
the flow rate of the stream of liquid through the heat transferer (103, 203, 303, 403) in the primary circuit (102, 202, 302, 402) and/or a flow rate of the heat transfer medium through the heat transferer in the secondary circuit (104, 204, 304, 404) is/are manipulated on the basis of the control signal.

### DESCRIPTION

The invention relates to a method of closed-loop control of the temperature in a chemical engineering apparatus, in which, in a primary circuit, a liquid is conveyed out of the apparatus, fed at least partly to a heat transferer and recycled at least partly back to the apparatus.

One prior publication that describes known methods of closed-loop control of the temperature in a chemical engineering apparatus is as follows, on pages 165-171: H. Schuler, Prozessführung [Process Engineering], Oldenbourg Verlag Munich Vienna, 1999. In this context, for example, temperatures are ascertained by sensors, or flow rates by valve settings or flow sensors, and used as controller input signals for the closed-loop control method.

For closed-loop control of the temperature in a reactor, there are different known concepts based on heat transfer elements in the reactor. Such heat transfer elements may be pipe coils inter alia, or comprise a cooling or heating jacket on the outside of the reactor. In the case of some exothermic reactions, however, a cooling jacket is insufficient to remove the heat released in the reaction. In these cases, the reaction mixture is typically conveyed from the reactor into a primary circuit comprising a heat transferer and a recycling conduit into the reactor. The heat transferer removes the heat of reaction from the reaction mixture. One document that discloses details of the use, design and mode of function of heat transferers is as follows, in chapter 8.29: B. G. Liptak, Process Control and Optimization, Instrument Engineers' Handbook, Volume Two, Taylor & Francis Inc., 4th edition, 2005.

In a specific application for hydroformylations, document WO 2015/047723 A1 (THE DOW CHEMICAL COMPANY) discloses a temperature closed-loop control system for a reactor, in which the temperature in the reactor is detected by a temperature sensor and a closed-loop control algorithm adjusts the flow rate through a heat transferer in a primary circuit such that the reactor temperature is kept at its target value. In an alternative variant, the primary circuit comprises a bypass parallel to the heat transferer, such that the stream in the primary circuit can be divided into two parallel substreams. The reactor temperature in this case too can be controlled by influencing the flow through the bypass.

Disadvantages of these disclosed temperature closed-loop control systems are the nonlinear control characteristics, the poor control quality and fluctuation characteristics that occur in dynamic processes, for example a change in load over time or the effect of a perturbation parameter.

The problem addressed was therefore that of providing a more stable and precise closed-loop control method for the temperature in a chemical engineering apparatus. It was also the intention in this context to improve the control quality in the event of dynamic changes in load and perturbations in the process.

This object was achieved in accordance with the invention by a method according to claim 1. Advantageous configurations of the method of the invention are specified in claims 2 to 12.

The method of the invention for closed-loop control of the temperature in a chemical engineering apparatus, in which, in a primary circuit, a liquid is conveyed out of the apparatus, fed at least partly to a heat transferer and recycled at least partly back to the apparatus, where the heat transferer is cooled or heated by a heat transfer medium in a secondary circuit, comprises the steps of:
providing a target value for the temperature of the liquid in the apparatus,
detecting an actual value for the temperature of the liquid in the apparatus,
calculating the temperature difference between the actual value and the target value of the liquid in the apparatus.

According to the invention,
a heat flow taken from or added to the liquid in the primary circuit by the heat transferer is ascertained,
a control signal is calculated on the basis of a defined closed-loop control algorithm, where the closed-loop control algorithm is configured such that the control signal is dependent on the heat flow and the temperature difference between the actual value and the target value of the liquid in the apparatus, and
the flow rate of the stream of liquid through the heat transferer in the primary circuit and/or a flow rate of the heat transfer medium through the heat transferer in the secondary circuit is/are manipulated on the basis of the control signal.

It has been found that the inclusion of the heat flow in the closed-loop control of the temperature in a chemical engineering apparatus enables both more stable closed-loop control and better control quality.

The closed-loop control algorithm is configured such that the control signal is dependent on the heat flow and the temperature difference between the actual value and the target value of the liquid in the apparatus. In the context of this document, this has the same meaning as the calculating of the control signal with inclusion of the heat flow and the temperature difference between the actual value and the target value of the liquid in the apparatus. In relation to the ascertaining of the control signal based on the defined closed-loop control algorithm, the terms “calculated” and “generated” are used synonymously in this document.

The terms “included” and “inclusion” in relation to the closed-loop control algorithm should be understood in this document such that an output parameter calculated by the closed-loop control algorithm, for example a control signal, is dependent on the respective parameter included. These terms should not be considered to be exclusive. The including of a parameter X does not mean that a parameter Y is not also included in the closed-loop control algorithm. For example, the including of the heat flow in the closed-loop control algorithm should be understood such that the control signal is calculated depending on the value of the heat flow, and the including of the temperature difference between the actual value and the target value of the liquid in the apparatus in the closed-loop control algorithm should be understood such that the control signal is calculated depending on the value of the temperature difference. The dependence may be expressed by way of example by a mathematical function f of the following form: “control signal=f(heat flow, temperature difference between the actual value and the target value of the liquid in the apparatus)”. Such a function may also comprise further parameters or influencing variables as well as the heat flow and the temperature difference, for example perturbation parameters.

A “chemical engineering apparatus” in this document is understood to mean an apparatus that can accept a volume of liquid and is suitable for a liquid to flow through, for example a reactor, a stirred vessel, a column, a phase separator or a tank. In preferred embodiments of the method of the invention, the chemical engineering apparatus is a reactor.

A “heat transferer” in this document is understood to be an apparatus that can transfer heat from one medium to another medium. By way of example, it is possible to use plate heat transferers or shell-and-tube heat transferers in the method of the invention.

A “primary circuit” in this document is a circulation stream in which a liquid is conveyed out of the chemical engineering apparatus, fed at least partly to a heat transferer and recycled at least partly back to the apparatus. The primary circuit may also comprise branches, for example in the form of a bypass parallel to the chemical engineering apparatus. It is also possible for multiple heat exchangers to be present in the primary circuit, for example as parallel interconnections, series interconnections or combinations of parallel and series interconnections.

A “closed-loop control algorithm” in the context of this document generates a control signal for manipulation of the flow rate of the liquid stream through the heat transferer in the primary circuit depending on one or more control variances, including the temperature difference between the actual value and the target value of the temperature of the liquid in the apparatus. According to the invention, the closed-loop control algorithm generates the control signal additionally depending on the heat flow removed or added by a heat transferer. The heat flow, just like the temperature difference between the actual value and the target value of the liquid in the apparatus, constitutes an influencing parameter for the closed-loop control algorithm. Further influencing parameters may be included in the closed-loop control algorithm for calculation of the control signal, for example flow rates of reactant or parameters that represent a chemical conversion in the apparatus.

The closed-loop control algorithm may result from direct electronic interconnection of hardware components. Examples of hardware components are PID controllers, temperature sensors, flow sensors and control units for adjustment of the flow rate of the heat transfer medium in the secondary circuit or the flow rate of the stream of the liquid in the primary circuit. The closed-loop control algorithm may additionally also result from interplay between hardware and software components, with the software components calculating one or more output signals on the basis of input parameters through computer-implemented numerical algorithms. The output signal may, by way of example, be the control signal for a control unit that adjusts, for example, the flow rate of the stream of the liquid.

The specific configuration, how the individual influencing parameters are correlated to one another and how the influencing parameters generate the control signal in detail may be described by, for example, a mathematical relationship, preferably by a mathematical algebraic function, or by partial or standard difference equations.

In many cases, chemical engineering processes are regulated and/or controlled by a process control system, which preferably use single-variable controllers. The single-variable controllers receive an input signal and give an output signal in accordance with their closed-loop control algorithm. Thus, a single-variable controller may preferably also be a PID controller.

The mathematical relationships for the PID controller are shown by way of example hereinafter.

$$y ( t ) = {K}_{p} ( e ( t ) + \frac{1}{{T}_{i}} {\int }_{0}^{t} e ( τ ) d τ ) + {T}_{d} \frac{de ( t )}{dt}$$

In this equation, y(t) is the controller output as a function of time t, e(t) is the control variance as a function of time t, Kₚ is the controller output, which represents the proportion of the controller function, Tᵢ is the integration time constant, which represents the integral of the controller function, τ is the integration variable, and T_{d} is the derivative of the time constant, which represents the difference of the controller function. The control variance e(t) is calculated as the difference between the target value w(t) and the actual value x(t) as e(t)=w(t)−x(t).

The closed-loop control algorithm may also be an adaptive closed-loop control algorithm. For example, the closed-loop control algorithm may be configured such that a parameter of the closed-loop control algorithm, for example the controller gain Kₚ, is not fixed but calculates its value over time as a function of particular process parameters. For example, the controller gain Kₚ may be formulated as a function of the heat capacity flow on the primary side and/or the secondary side of the heat exchanger, in order to compensate for nonlinearities in the heat transfer characteristics. Nonlinearities for the different flow regimes can be found in known specialist literature, for example in the VDI Heat Atlas (VDI-Wärmeatlas, Springer Vieweg, Berlin, Heidelberg, 12th edition, 2019, ISBN 978-3-662-52988-1, eBook: 978-3-662-52989-8).

A “perturbation parameter” in this document is understood to mean a measured or estimated process parameter that has a perturbing effect on the parameter to be controlled and is not affected by the closed-loop control algorithm. For example, this may be a raw material stream in the chemical engineering apparatus or an added or removed heat flow.

A “perturbation parameter feedforward” in this document means that the closed-loop control algorithm includes a perturbation parameter in the calculation of a control signal.

A “liquid” in this document is understood to mean a monophasic or polyphasic fluid. The liquid is thus free-flowing and conveyable through the primary circuit. The liquid may also comprise gas components and/or solid components, provided that such a liquid is still conveyable.

The term “liquid flow” in this document is understood to mean “flow of the liquid” in the sense of “flow rate of the flow of the liquid”, unless a different definition is apparent from the context. Therefore, these three terms are used interchangeably in this document.

A “heat transfer medium” in this document is understood to mean a monophasic or polyphasic fluid, for example a liquid, a gas, a vapor or mixtures thereof. The heat transfer medium is thus free-flowing and is conveyable through the secondary circuit. The heat transfer medium may also comprise solid components, provided that such a heat transfer medium is still conveyable. In some applications, the heat transfer medium is preferably heating steam, air or filtered river water.

In a preferred configuration of the method of the invention, in the closed-loop control algorithm, a heat flow difference between the heat flow ascertained as actual value and a target value of the heat flow which is fixed or defined in a variable manner over time is ascertained. On the basis of this difference in heat flow and the temperature difference ascertained between the actual value and the target value of the temperature of the liquid in the apparatus, a control signal is calculated, on the basis of which the flow rate of the stream of liquid through the heat transferer in the primary circuit and/or a flow rate of the heat transfer medium through the heat transferer in the secondary circuit is manipulated.

In a preferred configuration of the method of the invention, the heat flow is ascertained from the liquid flow through the heat transferer in the primary circuit and from a temperature difference between the temperature of the liquid upstream and downstream of the heat transferer in the primary circuit. In an alternative preferred configuration, the heat flow is ascertained from a flow rate of the heat transfer medium through the heat transferer in the secondary circuit and from a temperature difference between the temperature of the heat transfer medium upstream and downstream of the heat transferer in the secondary circuit. These two preferred configurations may also be combined, such that the heat flow is ascertained from a liquid flow through the heat transferer in the primary circuit and from a temperature difference between the temperature of the liquid upstream and downstream of the heat transferer in the primary circuit, and from a flow rate of the heat transfer medium through the heat transferer in the secondary circuit and from a temperature difference between the temperature of the heat transfer medium upstream and downstream of the heat transferer in the secondary circuit.

The ascertaining of the heat flow in this configuration has the advantage of easy implementability in terms of measurement technology. Proven and reliable sensors for flow and temperature are commercially available for different applications, and so, according to this configuration of the method of the invention, it is possible to implement simple and reliable ascertaining of the heat flow. Moreover, the detecting of the flows and temperatures used for ascertainment in the vicinity of the heat transferer makes it possible to rapidly detect any perturbations or fluctuations that occur there and take them into account in the closed-loop temperature control before the perturbations or fluctuations affect the temperature in the chemical engineering apparatus.

In a further preferred configuration of the method of the invention, the closed-loop control algorithm comprises a temperature controller and a heat flow controller, where the output signal calculated by the temperature controller is dependent on the temperature difference between the actual value and the target value of the liquid in the apparatus and is a target value for the heat flow taken from or added to the liquid in the primary circuit through the heat transferer, this target value is transmitted to the heat flow controller, and the output signal generated by the heat flow controller is dependent on the difference between the actual value and the target value of the heat flow and is the control signal for manipulation of the liquid flow through the heat transferer in the primary circuit and/or for manipulation of the flow rate of the heat transfer medium through the heat transferer in the secondary circuit. This configuration of the closed-loop control algorithm corresponds to a cascade closed-loop controller with the temperature controller as master controller and the heat flow controller as follower controller.

In an advantageous configuration of cascade closed-loop control, the master controller and the follower controller are each designed as PID controllers. In order to form the cascade, the controller output y₁(t) from the master controller is set equal to the target value w₂(t) for the follower controller. The actual values are calculated from the measurement of the temperature in the apparatus and the heat flow ascertained.

In a particularly preferred configuration of cascade closed-loop control, the following equations are implemented in the master controller and the follower controller:

$$\\ {y}_{1} ( t ) = {w}_{2} ( t ) = {\dot{Q}}_{HE , target} = {K}_{P , 1} ( {T}_{R , target} ( t ) - {T}_{R} ( t ) + \frac{1}{{T}_{i , 1}} {\int }_{0}^{t} ( {T}_{R , target} ( τ ) - {T}_{R} ( τ ) ) d τ ) + {T}_{d , 1} \frac{d ( {T}_{R , target} ( t ) - {T}_{R} ( t ) )}{dt}$$

$$\\ {y}_{2} ( t ) = {\dot{m}}_{HE , target} = {K}_{P , 2} ( {\dot{Q}}_{HE , target} ( t ) - {\dot{Q}}_{HE} ( t ) + \frac{1}{{T}_{i , 2}} {\int }_{0}^{t} ( {\dot{Q}}_{HE , target} ( τ ) - {\dot{Q}}_{HE} ( τ ) ) d τ ) + {T}_{d , 2} \frac{d ( {\dot{Q}}_{HE , target} ( t ) - {\dot{Q}}_{HE} ( t ) )}{dt}$$

In these equations, \dot{Q}_{HE,target} is the target value of the heat flow which is determined by the master controller, K_{P,1} is the controller gain of the master controller for the temperature of the liquid in the apparatus, T_{R,target} is the target value of the temperature of the liquid in the apparatus, T_{R} is the actual value of the temperature of the liquid in the apparatus, T_{i,1} is the integrated time constant of the master controller for the temperature of the liquid in the apparatus, T_{d,1} is the derivative of the time constant of the master controller for the temperature of the liquid in the apparatus, \dot{m}u_{HE,target} is the target value of the controlled mass flow rate through the heat transferer, K_{P,2} is the controller gain of the follower controller for the heat flow, \dot{Q}_{HE} is the actual value of the heat flow, T_{i,2} is the integrated time constant of the follower controller for the heat flow, and T_{d,2} is the derivative of the time constant of the follower controller for the heat flow.

It has been found that the heat flow added to or removed from the liquid in the primary circuit by the heat transferer reacts more quickly to control interventions by the closed-loop control algorithm or perturbations than the temperature measurable in the chemical engineering apparatus. A cascade arrangement of temperature controller as master controller and heat flow controller as follower controller has the advantage that the differences in the dynamics of the control circuit components are utilized in order to more quickly and precisely adjust the target temperature in the chemical engineering apparatus in the event of changes in target value or perturbations. The control quality of the closed-loop control algorithm is thus improved.

In a preferred configuration, there is at least one bypass in the primary circuit that runs parallel to the heat transferer. This enables more flexible configuration of the liquid flow in the primary circuit. This is advantageous, for example, when there are technical or process-related restrictions that affect the liquid flow in the primary circuit. For example, when a cost-efficient air cooler is used as heat transferer, the cooling output can typically be adjusted only in stages or only to a constant value. In such a case in which the air cooler is operated constantly, a bypass in the primary circuit also has the advantage that the flow rate of the liquid through the heat transferer can be controlled in a very simple manner in that the temperature in the chemical engineering apparatus can be controlled by the manipulation of the liquid flow through the bypass.

The manipulation of the liquid flow through the heat transferer in the primary circuit can be implemented in different ways, for example directly, indirectly or via a combination of direct and indirect manipulation.

In the case of direct manipulation of the liquid flow through the heat transferer, there is preferably at least one control unit and/or a pump for manipulation of the liquid flow on the basis of the control signal in the primary circuit in the inlet to the heat transferer or in the outlet from the heat transferer, or both in the inlet to the heat transferer and in the outlet from the heat transferer.

In the case of indirect manipulation of the liquid flow through the heat transferer, there is preferably at least one control unit and/or a pump for manipulation of the liquid flow through the heat transferer on the basis of the control signal in a bypass parallel to the heat transferer.

In the case of a combination of direct and indirect manipulation, there is preferably at least one control unit for manipulation of the liquid flow on the basis of the control signal in a bypass parallel to the heat transferer. In addition, in this variant, there is at least one control unit for manipulation of the liquid flow on the basis of the control signal in the primary circuit in the inlet to the heat transferer or in the outlet from the heat transferer, or both in the inlet to the heat transferer and in the outlet from the heat transferer.

For manipulation of the flow rate of the heat transfer medium through the heat transferer in the secondary circuit on the basis of the control signal, there is preferably at least one control unit in the inlet to the heat transferer or in the outlet from the heat transferer, or both in the inlet to the heat transferer and in the outlet from the heat transferer.

Suitable control units are known to the person skilled in the art. The control unit is preferably a controller valve suitable for rapid and precise adjustment of a flow rate.

According to the invention, a heat flow is removed from or added to the liquid in the primary circuit by a heat transferer. In addition, the liquid flow through the heat transferer and/or the flow rate of the heat transfer medium in the secondary circuit is manipulated by a control signal. However, the invention is not limited to exactly one heat transferer. The invention also covers embodiments in which there are two or more heat transferers in the primary circuit. These may be arranged in parallel interconnections, series interconnections, or combinations of parallel and series interconnections. In this case, the liquid flow is manipulated by at least one heat transferer. However, it is also possible for liquid flows to be manipulated by multiple heat transferers by the method of the invention.

In one configuration, there is a further heat transferer in the primary circuit parallel to the first heat transferer, such that the temperature of the chemical engineering apparatus can be controlled by two heat transferers if required. Such a configuration increases flexibility with regard to the provision of heating output or cooling output. A use example in which this flexibility can be exploited in an economically advantageous manner is the integration of heating output or cooling output from other process stages or other plants that would otherwise remain unutilized and cannot be influenced within the scope of the closed-loop control of the invention.

In a further configuration, there is at least one control unit and/or a pump for manipulation of the liquid flow on the basis of the control signal in the primary circuit in the inlet to the first and to the second heat transferer.

In a further configuration, there is at least one control unit and/or a pump for manipulation of the liquid flow on the basis of the control signal in the primary circuit in the inlet to the first and to the second heat transferer and in a bypass connected in parallel to the heat transferers.

In the configurations which have a control unit in a bypass and/or which have at least two heat transferers with their respective control units, the liquid flow can be limited by the respective heat transferer. This is an advantage particularly when, for example, a pump for the primary circuit is capable of conveying only a constant liquid flow.

In a configuration in which the liquid flow in the primary circuit is conveyed by a pump with a variable delivery rate, this pump can be used advantageously for manipulation of the flow rate of the stream of the liquid through the heat transferer on the basis of the control signal, for example in that the control signal defines the speed of the pump.

In a further configuration, the closed-loop control algorithm comprises a split range control function where, rather than one control signal, at least two control signals are calculated and at least two streams in the primary circuit and/or in the secondary circuit are manipulated, where a first stream is manipulated as a function of a first control signal and a second stream as a function of a second control signal.

In a further configuration, the closed-loop control algorithm comprises a split range control function where at least two control signals are calculated and at least two streams in the primary circuit are manipulated, where a first liquid flow is manipulated as a function of a first control signal and a second liquid flow as a function of a second control signal.

In a further configuration, the closed-loop control algorithm comprises a split range control function where at least two control signals are calculated and at least two streams in the secondary circuit are manipulated, where a first flow rate is manipulated as a function of a first control signal and a second flow rate as a function of a second control signal.

In a further configuration, the closed-loop control algorithm comprises a split range control function where at least two control signals are calculated and at least two streams, one of which is in the primary circuit and one in the secondary circuit, are manipulated, where a liquid stream in the primary circuit is manipulated as a function of a first control signal and a flow rate in the secondary circuit as a function of a second control signal.

Configurations with split range control functions are advantageous especially when there are control units with different nominal widths and hence significantly different flow rates. In such a case, precise control of the flow rate of the heat transfer medium can be ensured in that a large control unit designed for large flow rates and a small control unit designed for small flow rates are arranged in a parallel interconnection.

In a preferred configuration of the method of the invention, in which there is at least one bypass parallel to a heat transferer in the primary circuit, the amount of liquid being conveyed out of the chemical engineering apparatus into the primary circuit is set to a defined target value by ascertaining the actual value of the amount of liquid, comparing the actual value and target value to calculate an output signal, and manipulating the liquid flow flowing through the bypass using the output signal. This configuration is advantageous especially when the liquid flow being conveyed out of the chemical engineering apparatus into the primary circuit is to be or has to be kept constant, but the aim at the same time is efficient closed-loop control of the temperature in the apparatus.

In a further configuration, the closed-loop control algorithm includes at least one perturbation parameter in the calculation of the control signal, where the perturbation parameter comprises a measured or estimated process parameter. For example, the perturbation parameter may be a raw material stream which is guided into the chemical engineering apparatus. The perturbation parameter may also, for example, comprise one or more further heat flows. One resultant advantage here is that the closed-loop controller is able to react in good time in the event, for example, of a forthcoming change in the heat released or consumed in the apparatus owing to altered input flows. It is also possible to take note of multiple or combined perturbation parameters.

In a further configuration, a chemical or biological, endothermic or exothermic reaction or an exothermic or endothermic physical process is proceeding in the chemical engineering apparatus.

In a preferred configuration, the reaction is a hydroformylation, etherification, ether cleavage, enalization, dehydrogenation, pyrolysis, hydrogenation, or a cracking process.

In a particularly preferred configuration, the hydrogenation is a full hydrogenation, selective hydrogenation or ring hydrogenation.

In the case of such reactions and processes, major changes in load or influences can result from perturbation parameters. The method of the invention, especially in the case of such demanding closed-loop control tasks, assures rapid and robust establishment of the desired temperature in the chemical engineering apparatus.

In a further configuration, the closed-loop control algorithm is a multiparameter closed-loop controller, preferably a model-predictive closed-loop controller or a closed-loop state controller. One resultant advantage here is that multiple physical parameters can be detected simultaneously in the method and used for closed-loop control.

The invention will be elucidated in detail hereinafter with reference to the drawings. The drawings should be considered to be schematic diagrams. They do not constitute a limitation of the invention, for example with regard to specific dimensions or design variants. The figures show:

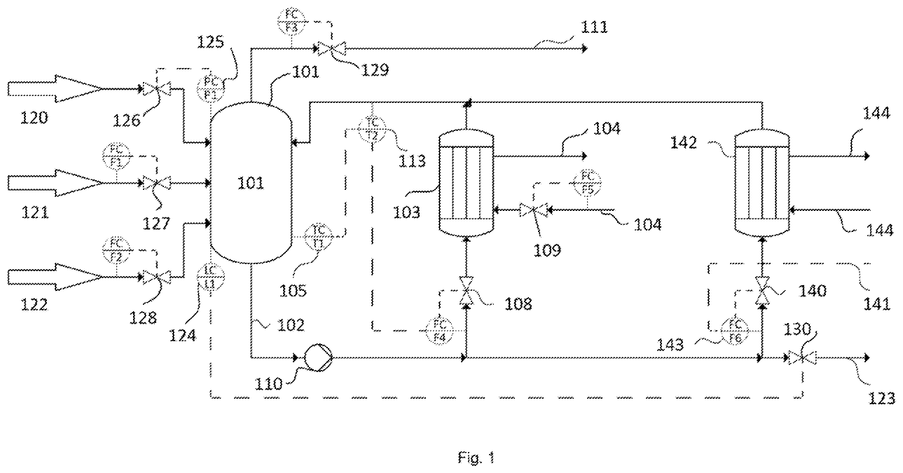

FIG. 1: Chemical engineering apparatus with external heat transferers and closed-loop control structure for closed-loop control of the reactor temperature according to the prior art

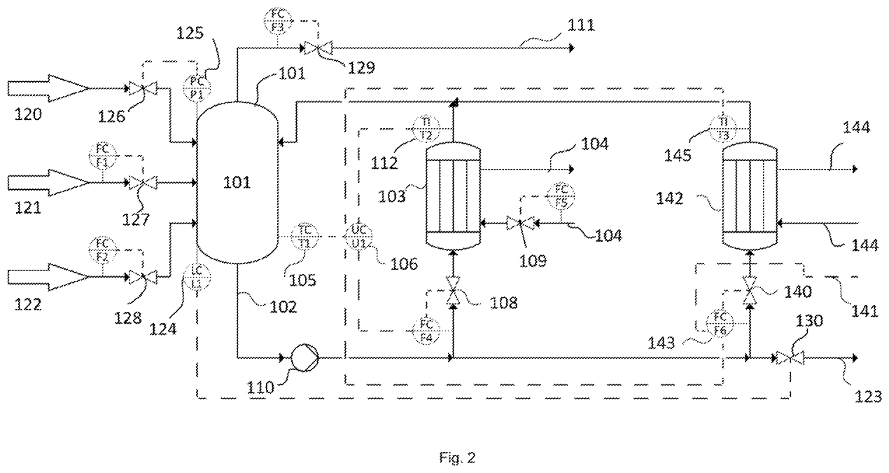

FIG. 2: Chemical engineering apparatus with external heat transferers and closed-loop control structure for closed-loop control of the reactor temperature according to a first embodiment of the invention

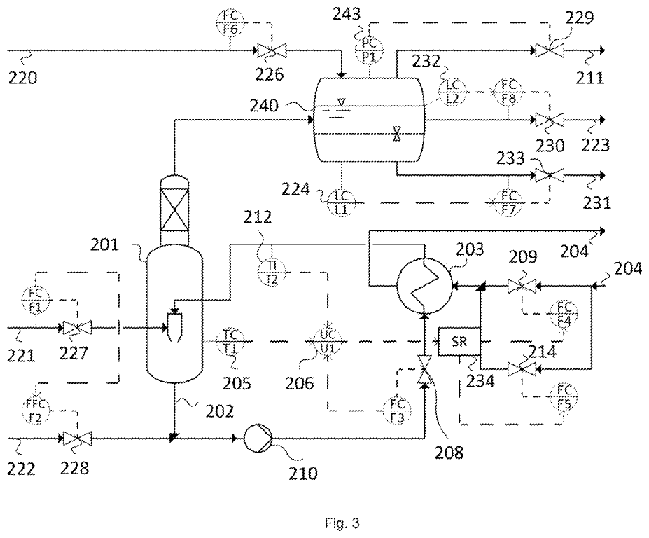

FIG. 3: Chemical engineering apparatus with external heat transferers and closed-loop control structure for closed-loop control of the reactor temperature according to a second embodiment of the invention

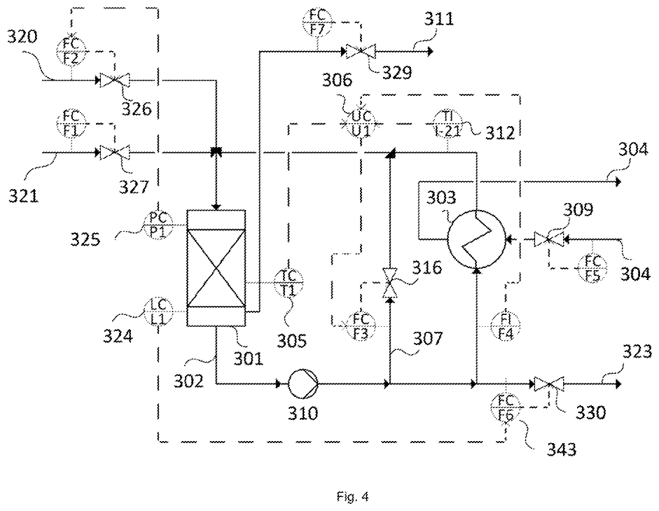

FIG. 4: Chemical engineering apparatus with external heat transferers and closed-loop control structure for closed-loop control of the reactor temperature according to a third embodiment of the invention

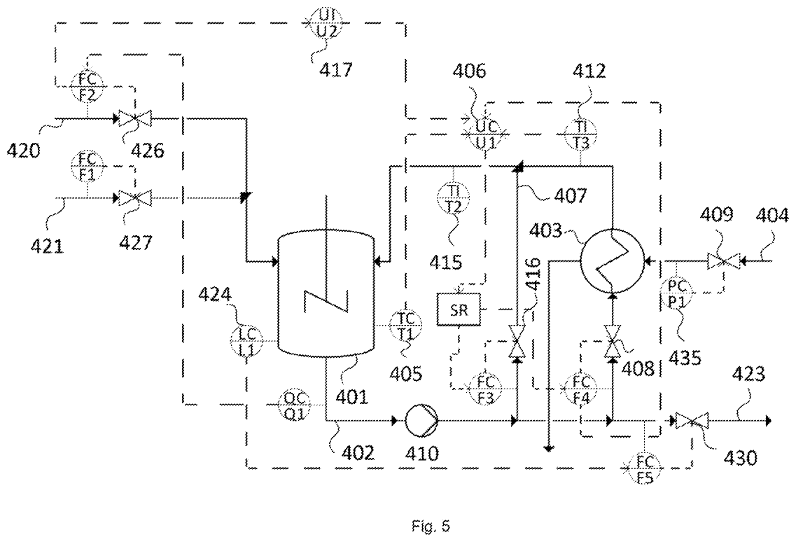

FIG. 5: Chemical engineering apparatus with external heat transferers and closed-loop control structure for closed-loop control of the reactor temperature according to a fourth embodiment of the invention

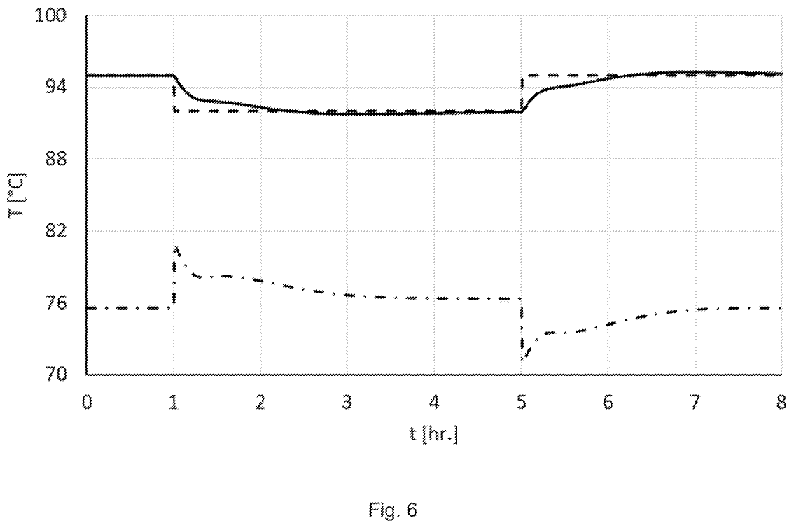

FIG. 6: Control characteristics in the event of a change in target value by 1° C. for example 1 in the case of prior art closed-loop control with temperature-temperature cascade

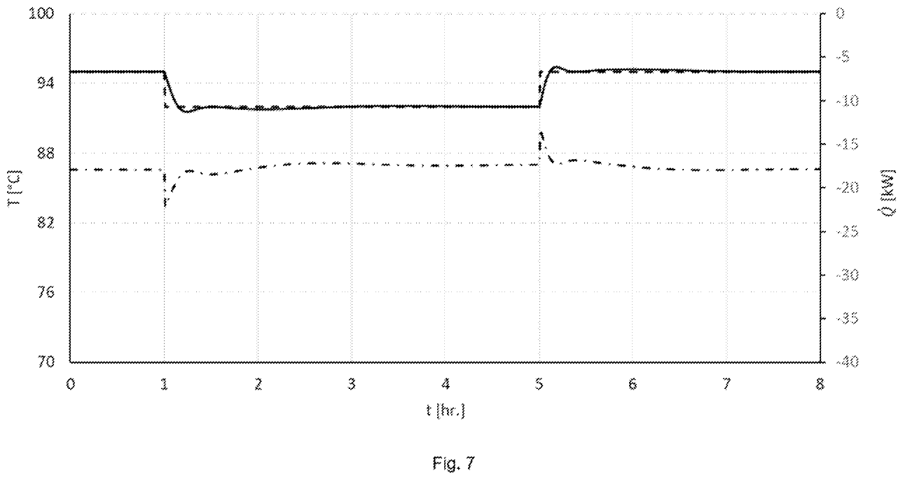

FIG. 7: Control characteristics in the event of a change in target value by 1° C. for example 1 in the case of inventive closed-loop control with temperature-heat flow cascade

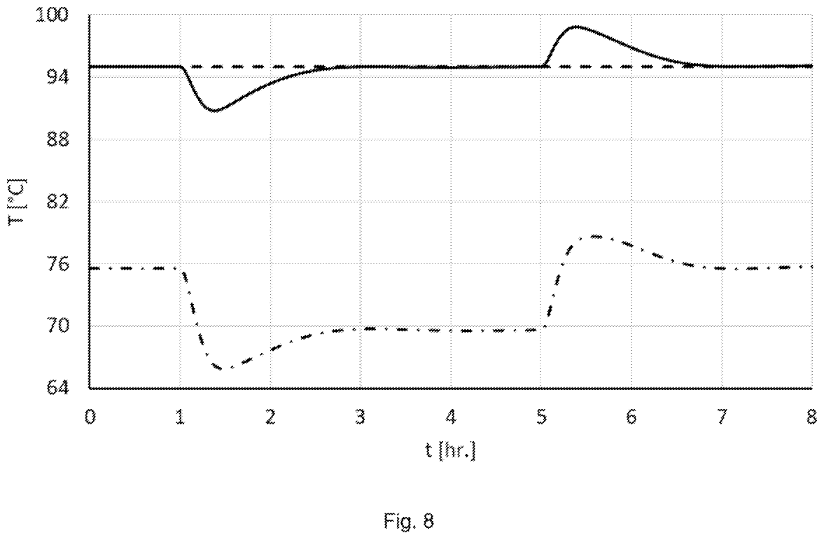

FIG. 8: Perturbation characteristics in the event of a change in propene feed by 10% for example 1 in the case of prior art closed-loop control with temperature-temperature cascade

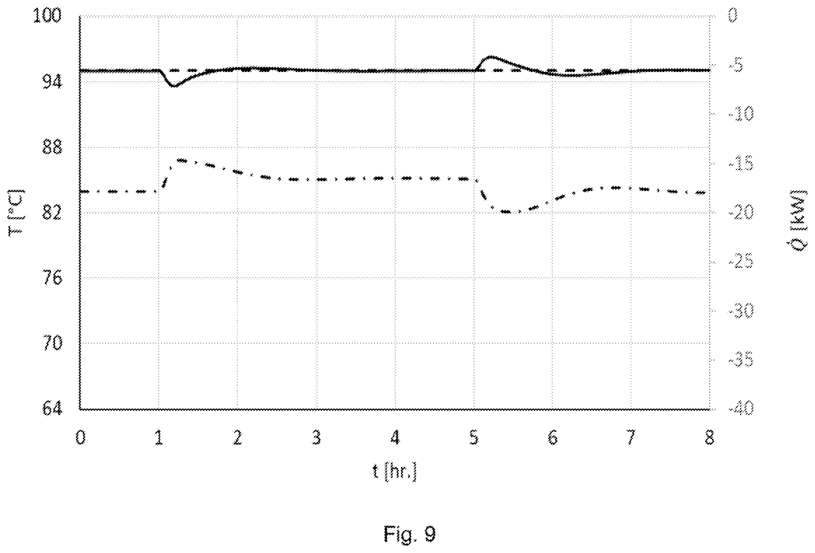

FIG. 9: Perturbation characteristics in the event of a change in propene feed by 10% for example 1 in the case of inventive closed-loop control with temperature-heat flow cascade

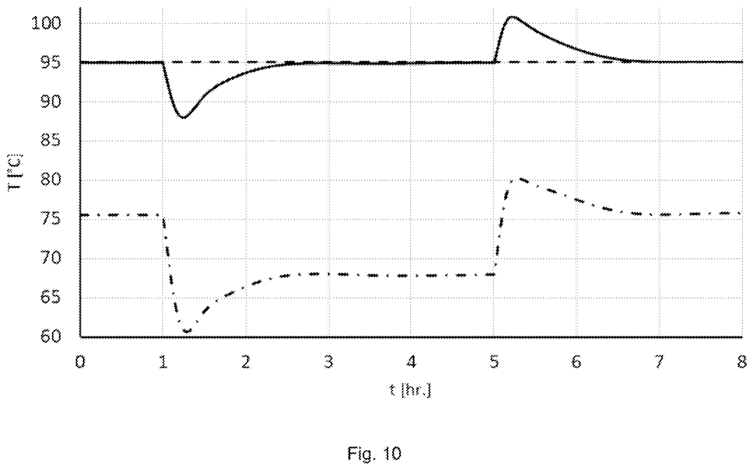

FIG. 10: Perturbation characteristics in the event of a change in the heat flow to be removed by heat integration by 10% for example 1 in the case of prior art closed-loop control with temperature-temperature cascade

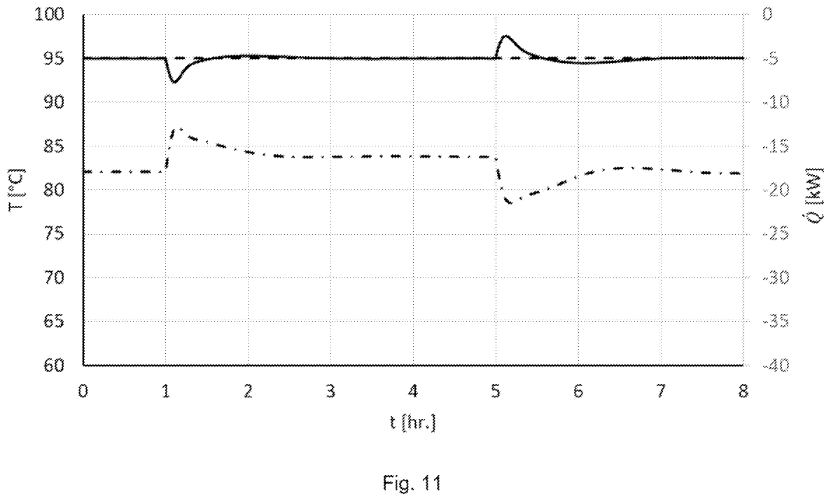

FIG. 11: Perturbation characteristics in the event of a change in the heat flow to be removed by heat integration by 10% for example 1 in the case of inventive closed-loop control with temperature-heat flow cascade

### LIST OF REFERENCE NUMBERS USED

101 chemical engineering apparatus
102 primary circuit
103 heat transferer
104 secondary circuit
105 temperature controller with temperature sensor in the chemical engineering apparatus
106 follower heat flow controller
108, 109 control unit
110 pump
111 conduit for material recovery
112 temperature sensor in the recycling conduit of the primary circuit to the chemical engineering apparatus
113 follower temperature controller including a temperature sensor in the return conduit of the heat transferer
120 conduit (synthesis gas)
121 conduit (propene)
122 conduit (solvent)
123 conduit for the discharge of the target product
124 controller for the liquid level in the chemical engineering apparatus
125 pressure controller
126, 127, 128, 129, 130, 140 control unit
141 external definition of a pressure signal
142 heat transferer
143 flow controller for the adjustment of the liquid flow through the heat transferer
144 secondary circuit
145 temperature sensor downstream of the heat transferer
201 chemical engineering apparatus
202 primary circuit
203 heat transferer
204 secondary circuit
205 temperature controller with temperature sensor in the chemical engineering apparatus
206 follower heat flow controller
208 control unit
209 large control unit for adjustment of the heat transfer medium flow through the heat transferer
210 pump
211 conduit (offgas)
212 temperature sensor in the return conduit of the heat transferer
214 small control unit
220 conduit for inert gas (nitrogen)
221 conduit (aldehyde)
222 conduit (water with catalyst)
223 conduit for the discharge of the target product
224 controller for the liquid level (water) in the phase separator
226, 227, 228, 229, 230 control unit
231 conduit for the wastewater
232 controller for the liquid level (target product) in the phase separator
233 control unit
234 split range controller
240 phase separator
243 pressure regulator for the closed-loop control of the offgas stream
301 chemical engineering apparatus
302 primary circuit
303 heat transferer
304 secondary circuit
305 temperature controller with temperature sensor in the chemical engineering apparatus
306 follower heat flow controller
307 bypass
309 control unit
310 pump
311 conduit for the offgas
312 temperature sensor in the return conduit of the heat transferer
315 temperature sensor directly upstream of the recycling of the liquid flow into the chemical engineering apparatus
316 control unit
320 conduit (hydrogen)
321 conduit (alkene)
323 conduit (crude alcohol)
324 controller for the liquid level in the chemical engineering apparatus
325 pressure controller
326, 327, 329, 330 control unit
343 flow controller that defines the mass flow rate (crude alcohol)
401 chemical engineering apparatus
402 primary circuit
403 heat transferer
404 secondary circuit
405 temperature controller with temperature sensor in the chemical engineering apparatus
406 follower heat flow controller
407 bypass
408, 409 control unit
410 pump
412 temperature sensor in the return conduit of the heat transferer
415 temperature sensor immediately upstream of the recycling of the liquid flow into the chemical engineering apparatus
416 control unit
417 perturbation parameter calculation
420 conduit (ammonium nitrate)
421 conduit (water)
423 conduit for the discharge of the target product
424 controller for the liquid level in the chemical engineering apparatus
426, 427, 430 control unit
435 pressure regulator for the control of the heat transfer medium flow for the heat transferer

FIG. 1 shows a process flow diagram of a chemical engineering process according to the prior art, in which the temperature in a reactor 101 as chemical engineering apparatus is adjusted by means of external heat transferers 103, 142. The reactor 101 is supplied with feedstocks via separate conduits that are converted to one or more products in the reactor. In the example described, a gaseous feedstock is fed in via conduit 120, and liquid or gaseous feedstocks via conduits 121 and 122. The feedstocks are fed in under flow rate control in conduits 121 and 122 by means of two control valves 127, 128 in the feed conduits. The amount of gaseous feedstock fed in in conduit 120 is adjusted via a pressure controller 125 and a control valve 126 in the feed conduit such that a defined pressure in the reactor 101 is maintained.

Gaseous reaction products and any unconverted gaseous feedstocks are removed from the reactor via a gaseous reactor outlet 111. In the example described, the discharge is effected under flow rate control via a control valve 129 in the reactor outlet 111. Liquid reaction products are discharged by forced conveying by means of a pump 110 into a primary circuit 102 which connects the reactor 101 to two external heat transferers 103, 142. The liquid stream conveyed out of the reactor 101 into the primary circuit 102 is divided between the two heat transferers and is adjustable via control units 108, 140 in the respective feed conduits to the heat transferers. The substreams from the primary circuit that have been heated or cooled in the heat transferers are returned to the reactor. A portion of the liquid stream is withdrawn as liquid reactor output 123 from the primary circuit 102. The withdrawal is effected by means of a control valve 130 in the withdrawal conduit and by means of a level controller 124 such that a defined liquid level in the reactor is maintained.

The temperature in the reactor 101 is set to a defined value. Depending on whether the reaction in the reactor proceeds endothermically or exothermically, heat has to be supplied to the reactor or removed from the reactor. The two heat transferers make different contributions in this regard. While the second heat transferer 142 bears a main load of the heat exchange, the first heat transferer 103 is used to control the temperature. The amount of the heat transfer medium in the secondary circuit 144 in the second heat transferer 142 is not under flow rate control, whereas the amount of the heat transfer medium in the secondary circuit 104 of the first heat transferer 103 is adjusted to a defined value by means of a control valve 109. The amount of liquid which flows into the second heat transferer in the primary circuit is adjusted in the example described via a flow controller 143 and the control valve 140 in the feed such that a temperature is maintained in a downstream process stage. This process stage is not shown in FIG. 1. The target parameter for the flow as controller output from the temperature controller in the process stage that is not shown is symbolized by the dotted line 141.

For closed-loop control of the temperature in the reactor 101, a cascade closed-loop controller is provided, comprising a first temperature controller 105 as master controller and a second temperature controller 113 as follower controller. A target value for the temperature of the liquid in the reactor 101 is set in the master controller 105. The actual value of the temperature in the reactor 101 is detected by means of a temperature sensor. Depending on the temperature difference between the actual value and the target value of the liquid in the reactor, the master controller 105 calculates, as output signal, a target value for the temperature of the liquid which is fed back to the reactor as return stream from the heat transferers in the primary circuit. This target value is transmitted to the follower controller 113, which uses the difference between the actual value and the target value of the temperature of the recycled liquid as output signal to generate a control signal for the control valve 108 in the feed to the first heat transferer.

FIG. 2 shows a schematic of a process flow diagram of a chemical engineering process according to a first embodiment of the method of the invention, in which the temperature in a reactor 101 is adjusted by means of external heat transferers 103, 142. The arrangement and interconnection of the apparatuses and the basic closed-loop control systems in the inlet and outlet conduits are identical to the method according to the prior art, and so reference is made in this regard to the above details relating to FIG. 1.

However, the method of the invention differs in the structure for control of the reactor temperature from the method according to the prior art. In the example shown in FIG. 2, for closed-loop control of the temperature in the reactor 101, a cascade closed-loop controller is provided, comprising a temperature controller 105 as master controller and a heat flow controller 106 as follower controller. A target value for the temperature of the liquid in the reactor 101 is set in the master controller 105. The actual value of the temperature in the reactor 101 is detected by means of a temperature sensor. Depending on the temperature difference between the actual value and the target value of the liquid in the reactor, the master controller 105 calculates, as output signal, a target value for the heat flow removed from or added to the liquid in the primary circuit 102 by the heat transferers 103 and 142. This target value is transmitted to the follower heat flow controller 106, which uses the difference between the actual value and the target value of the heat flow as output signal to generate a control signal for the control valve 108 in the feed to the first heat transferer.

In the example shown in FIG. 2, the heat flow removed from or added to the liquid in the primary circuit 102 by the heat transferers 103 and 142 is calculated from a proportion of the first heat transferer 103 and a proportion of the second heat transferer 142. The proportion of the first heat transferer 103 is ascertained from the liquid flow flowing through the control valve 108 in the feed to the heat transferer and from the temperature difference between the temperature of the liquid upstream and downstream of the heat transferer 103 in the primary circuit. The temperature upstream of the heat transferer which is used is the actual value of the reactor temperature 105. The temperature downstream of the heat transferer is ascertained by means of a temperature sensor 112 in the outlet from the heat transferer. Analogously, the proportion of the second heat transferer 142 is ascertained from the liquid flow flowing through the control valve 140 in the feed to the heat transferer and from the temperature difference between the temperature of the liquid upstream and downstream of the heat transferer 142 in the primary circuit. The temperature upstream of the heat transferer which is used is the actual value of the reactor temperature 105. The temperature downstream of the heat transferer is ascertained by means of a temperature sensor 145 in the outlet from the heat transferer.

The closed-loop control of the process via the heat flow leads to distinctly better control quality compared to the closed-loop control method known from the prior art, as apparent from comparative example 1 and example 1 that are reported below.

In one variant of the first embodiment of the method of the invention according to FIG. 2, perturbation parameters are included in the calculation of the control signal for the control valve 108. In the example shown, the perturbation parameters are changes in the composition and/or in the volume of the feedstocks. The flow rates of the feedstocks are detected in the control valves 126, 127 and 128. Thus, both the absolute amounts and their ratios to one another are known. The measured values of these measured process parameters are fed to the heat flow controller 106. Knowledge of the perturbation parameters and inclusion thereof in the calculation of the control signal for the control valve 108 in the feed to the first heat transferer 103 enables even faster reaction to control variances and hence even better control quality.

FIG. 3 shows a schematic of a process flow diagram of a chemical engineering process according to a second embodiment of the method of the invention, in which the temperature in a reactor 201 as chemical engineering apparatus is adjusted by means of an external heat transferer 203. The reactor 201 is supplied via a conduit with a liquid feedstock 221 which is converted to one or more products in the reactor. The supply is effected here under flow rate control by a control valve 227. In a defined ratio to liquid stream fed in, a further liquid stream with catalyst 222 is added, which is also under flow rate control by means of a control valve 228.

The liquid product formed in the reactor 201 is drawn off together with the liquid 222 having added catalyst in the upper region of the reactor 201, and fed to a phase separator 240.

In order to avoid possible formation of an explosive atmosphere in the phase separator 240, an inert gas 220 is conveyed into the phase separator 240 under flow rate control. The pressure in the phase separator 240 is controlled via the removal of offgas 232 by a control valve 229 which receives its control signal from the gas pressure controller 243 in the phase separator 240. The offgas here may comprise inert gas, nitrogen by way of example, and any gaseous or evaporated reaction products.

The level of the phase interface between the two liquid phases is controlled via the removal of the wastewater 231 by a fill level controller 224. The upper overall level in the vessel is controlled via the product removal of the liquid product 223. Closed-loop control is effected here by means of a fill level controller 232, the control signal from which controls the control valve 230.

A substream of the liquid phase present in the reactor 201 is discharged by forced conveying by means of a pump 210 into a primary circuit 202 that connects the reactor 201 to an external heat transferer 203. The flow rate conveyed from the reactor 201 into the primary circuit 202 is adjustable by means of the control valve 208. The stream heated or cooled in the heat transferer 203 is returned to the reactor 201 in the primary circuit.

In the example described, the heat flow removed from or added to the liquid in the primary circuit 202 by the heat transferer 203 is calculated. The heat flow removed or added in the heat transferer 203 is ascertained from the liquid flow flowing through the control valve 208 in the feed to the heat transferer and from the temperature difference between the temperature of the liquid upstream and downstream of the heat transferer 203 in the primary circuit. The temperature upstream of the heat transferer which is used is the actual value of the reactor temperature 205. The temperature downstream of the heat transferer is ascertained by means of a temperature sensor 212 in the outlet from the heat transferer.

In the example described, for closed-loop control of the temperature in the reactor 201, a cascade closed-loop controller is provided, comprising the temperature controller 205 as master controller and a heat flow controller 206 as follower controller. A target value for the temperature of the liquid in the reactor 201 is set in the master controller 205. The actual value of the temperature in the reactor 201 is detected by means of a temperature sensor. Depending on the temperature difference between the actual value and the target value of the liquid in the reactor 201, the master controller 205 calculates, as output signal, a target value for the heat flow removed from or added to the liquid in the primary circuit 202 by the heat transferer 203. This target value is transmitted to the follower heat flow controller 206, which uses the difference between the actual value and the target value of the heat flow as output signal to generate a control signal for the control valve 208 in the feed to the heat transferer 203.

The closed-loop control algorithm comprises a split range control function where two control signals are calculated and two flow rates in the secondary circuit 204 are manipulated, where a first flow rate is manipulated as a function of a first control signal and a second flow rate as a function of a second control signal.

In this case, the flow rate in the secondary circuit 204 is divided into two flow rates in a mutually parallel arrangement, before these are combined again and flow through the heat transferer 203. The parallel division of the flow rate allows one control unit for manipulation of the respective flow rate to be disposed in each of the two resulting flows. In this example, the two control units are given different nominal widths, which allows significantly different flow rates to be established. It is thus possible to assure precise control of the flow rate of the heat transfer medium by designing the larger of the two control units for large flow rates, and designing the smaller of the two control units for small flow rates.

When river water, for example, is used as heat transfer medium in the secondary circuit 204, the river water is discharged from the system after passing through the heat transferer 204.

The second embodiment of the method of the invention as shown in FIG. 3 is suitable, for example, for methods of enalization of aldehydes. In such methods, free jet reactors 201 are typically used for reaction of aldehydes with water in the presence of a catalyst in order to obtain enals as target products. The production of enals produces heat, which is removed via the external heat transferer 203.

The liquid feedstock 221 supplied to the reactor 201 under flow rate control is an aldehyde. In a defined ratio to the aldehyde stream, the reactor 201 is supplied with an aqueous liquid stream 222 comprising a catalyst in the aqueous phase. The liquid enal product formed in the reactor 201 is drawn off together with the liquid 222 having added catalyst in the upper region of the reactor 201, and fed to a phase separator 240. For avoidance of possible formation of an explosive atmosphere, the phase separator 240 is supplied with nitrogen as inert gas 220. In the phase separator 240, the liquid phase comprising the catalyst is withdrawn via conduit 231. The enal target product is withdrawn via conduit 223 in the organic phase.

A substream of the liquid phase present in the reactor 201 is discharged as circulation stream via a pump 210 by forced conveying into a primary circuit 202. This liquid stream is chosen such that intensive mixing of the organic and aqueous phases is established in the reactor 201. This results from an input of power which is introduced into the liquid via the circulation. The input of power through the liquid stream into the reactor 201 is thus adjustable.

In this embodiment, the heat flow is advantageously controlled via the flow rate of the heat transfer medium in the secondary circuit 204.

The various control circuits are preferably controlled using PID controllers with controller parameters optimized to the desired steady state. The actual value of the heat flow removed in the heat transferer 203 is calculated using the measured value of the temperature Tᵢₙ in the reactor 201, the measured value of the temperature T_{out,1} 212 downstream of the heat transferer 203, the measured value of the mass flow rate \dot{m}₁ through the control valve 208 in the feed to the heat transferer 203, and the parameter of the heat capacity cₗ for the medium of the liquid flow according to the formula \dot{Q}₁=cₗ\dot{m}₁(Tᵢₙ−T_{out,1}).

FIG. 4 shows a schematic of a process flow diagram of a chemical engineering process according to a third embodiment of the method of the invention, in which the temperature in a reactor 301 as chemical engineering apparatus is adjusted by means of an external heat transferer 303.

The reactor 301 is supplied via a conduit with a liquid feedstock 321 which is converted to one or more products in the reactor. The supply is effected here under flow rate control by a control valve 327.

A gas stream 320 is added to the reactor 301, which is under flow rate control by means of a control valve 326. The control valve 326 receives its control signal here from a pressure controller 325 that controls the pressure in the reactor. By means of a gaseous output 311, gaseous by-products are removed from the reactor under flow rate control by a control valve 329.

The liquid product formed in the reactor 301 is conveyed by means of a circulation pump 310 out of the reactor 301 into a primary circuit 302 that connects the reactor 301 to an external heat transferer 303.

The liquid flow through the heat transferer 303 is also adjustable indirectly by means of a control valve 316 in a bypass 307 in the primary circuit. The bypass here is connected parallel to the heat transferer 303 in the primary circuit.

The stream heated or cooled in the heat transferer 303 and the stream through the bypass 307 are returned to the reactor 301 in the primary circuit.

A portion of the liquid stream is withdrawn as liquid reactor output 323 from the primary circuit 302. The withdrawal is effected by means of a flow controller 343 and a control valve 330 in the withdrawal conduit and by means of a level controller 324 such that a defined liquid level in the reactor is maintained.

In the example described, the heat flow removed from or added to the liquid in the primary circuit 302 by the heat transferer 303 is calculated from the liquid flow flowing through the heat transferer 303 and from the temperature difference between the temperature of the liquid upstream and downstream of the heat transferer 303 in the primary circuit. The temperature upstream of the heat transferer which is used is the actual value of the reactor temperature 305. The temperature downstream of the heat transferer is ascertained by means of a temperature sensor 312 in the outlet from the heat transferer.

In this inventive example, for closed-loop control of the temperature in the reactor 301, a cascade closed-loop controller is provided, comprising the temperature controller 305 as master controller and a follower heat flow controller 306. A target value for the temperature of the liquid in the reactor 301 is set in the temperature controller 305. The actual value of the temperature in the reactor 301 is detected by means of a temperature sensor 305. Depending on the temperature difference between the actual value and the target value of the liquid in the reactor 301, the follower heat flow controller 306 calculates, as output signal, a target value for the heat flow removed from or added to the liquid in the primary circuit 302 by the heat transferer 303. This target value is transmitted to the follower heat flow controller 306, which uses the difference between the actual value and the target value of the heat flow as output signal to generate a control signal for the control valve 316 in the bypass 307.

The amount of the heat transfer medium in the secondary circuit 304 of the heat exchanger 303 is quantitatively controlled by a control valve 309 and usually set to a fixed value. The heat transfer medium in the secondary circuit 304 is chosen according to the demands on the required cooling or heating output. In the case of cooling, preference is given to using river water or cooled water as heat transfer medium.

The third embodiment of the method of the invention shown in FIG. 4 is suitable, for example, for processes for hydrogenation of alkenes and aldehydes, selective hydrogenations such as the selective hydrogenation of butadiene, and etherifications. In such processes, fixed bed reactors 301 are typically used for conversion of the starting materials to the target products.

In the example of a hydrogenation, the liquid feedstock to be hydrogenated is fed to the reactor 301 via the conduit 321 under flow rate control. Hydrogen is fed to the reactor 301 in gaseous form via the conduit 320 under pressure control. By means of a gaseous output 311, gaseous by-products are removed from the reactor under flow rate control by a control valve 329. The liquid product formed in the reactor 301, for example a butanol, is removed from the primary circuit via the conduit 323 under flow rate control.

The various control circuits are preferably controlled using PID controllers with controller parameters optimized to the desired steady state. The actual value of the heat flow removed in the heat transferer 303 is calculated using the measured value of the temperature Tᵢₙ in the reactor 301, the measured value of the temperature T_{out,1} 312 downstream of the heat transferer 303, the measured value of the mass flow rate \dot{m}₁ in the feed to the heat transferer 303, and the parameter of the heat capacity cₗ for the medium of the liquid flow according to the formula \dot{Q}₁=cₗ\dot{m}₁(Tᵢₙ−T_{out,1}).

FIG. 5 shows a schematic of a process flow diagram of a chemical engineering process according to a fourth embodiment of the method of the invention, in which the temperature in a reactor 401 is adjusted by means of an external heat transferer 403. The reactor 401 is supplied via a conduit with a first liquid feedstock 421 which is converted to one or more products in the reactor. The supply is effected here under flow rate control by a control valve 427. A second liquid feedstock 420 is added to the reactor 401, which is controlled by means of a control valve 426 on the basis of a measured molar concentration (Q1) in the liquid stream in the primary circuit 402. The molar concentration can also be ascertained via a substitute parameter, for example conductivity, density, viscosity or the speed of sound, provided that the substitute parameter enables conclusion of the molar concentration.

The liquid product formed in the reactor 401 is conveyed by means of a circulation pump 410 out of the reactor 401 into a primary circuit 402 that connects the reactor 401 to an external heat transferer 403. The flow rate through the heat transferer 403 is adjustable indirectly by means of a control valve 416 in a bypass 407, and directly by means of a control valve 408 upstream of the heat transferer 403.

The stream heated or cooled in the heat transferer 403 and the stream through the bypass 407 are returned to the reactor 401 in the primary circuit.

A portion of the liquid stream is withdrawn as liquid reactor output 423 from the primary circuit 402. The withdrawal is effected by means of a flow controller and a control valve 430 in the withdrawal conduit and by means of a level controller 424 such that a defined liquid level in the reactor is maintained.

In this example, the heat flow removed from or added to the liquid in the primary circuit 402 by the heat transferer 403 is calculated from the liquid flow flowing through the heat transferer 403 and from the temperature difference between the temperature of the liquid upstream and downstream of the heat transferer 403 in the primary circuit. The temperature upstream of the heat transferer which is used is the actual value of the reactor temperature 405. The temperature downstream of the heat transferer is ascertained by means of a temperature sensor 412 in the outlet from the heat transferer.

In this inventive example, for closed-loop control of the temperature in the reactor 401, a cascade closed-loop controller is provided, comprising the temperature controller 405 as master controller and a heat flow controller 406 as follower controller. A target value for the temperature of the liquid in the reactor 401 is set in the master controller 405. The actual value of the temperature in the reactor 401 is detected by means of a temperature sensor 405. Depending on the temperature difference between the actual value and the target value of the liquid in the reactor 401, the master controller 405 calculates, as output signal, a target value for the heat flow removed from or added to the liquid in the primary circuit 402 by the heat transferer 403. This target value is transmitted to the follower heat flow controller 406, which uses the difference between the actual value and the target value of the heat flow to calculate control signals. The closed-loop control algorithm of the follower controller 406 is designed as a split range controller in which two control signals are calculated. Depending on a first control signal, the liquid flow in the bypass 407 is manipulated by means of the control valve 416. Depending on a second control signal, the liquid flow in the primary circuit 402 is manipulated by the control valve 408 in the feed to the heat transferer 403. According to liquid flows to be established, the two control valves 408, 416 may independently be set between their closed positions and their open positions.

In the example shown in FIG. 5, the closed-loop control algorithm also takes account of perturbation parameters in the calculating of the control signal. A perturbation parameter known in the illustrative process is the influence of the amount of feedstock supplied on the evolution of heat in the reactor. Depending on whether the reaction proceeds exothermically or endothermically, the effect of feeding the second feedstock 420 to the first feedstock 421 is an increase or decrease in the temperature in the reactor 401. The positive or negative amount of heat that arises from the amount of second feedstock 420 supplied is calculated and fed forward to the follower controller 406 as perturbation parameter. The amount of heat can be calculated within the follower controller or, as shown in FIG. 5, in a separate model unit 417. The inclusion of the perturbation parameter makes the closed-loop control algorithm capable of reacting predictively to the expected change in temperature in the reactor 401 even before the change is apparent using the temperature sensor 405. In this way, it is possible to more quickly and better control changes in load, fluctuations or changes in target value in the process, which leads to improved control quality.

The flow rate of the heat transfer medium in the secondary circuit 404 of the heat transferer 403 is adjusted by means of a control valve 409 and a pressure controller 435 such that a defined pressure is maintained in the feed of the heat transfer medium to the heat transferer. This is advantageous especially when heating steam is used as heat transfer medium. The heat transfer medium in the secondary circuit 404 is chosen according to the demands on the required cooling or heating output. In the case of heating, preference is given to using heating steam as heat transfer medium in the secondary circuit 404.

The fourth embodiment of the method of the invention as shown in FIG. 5 is suitable, for example, for methods in which the chemical engineering apparatus 401 is a stirred vessel in which various substances are mixed with one another or one substance is to be dissolved in another substance.

In the example of the production of an aqueous solution of ammonium nitrate, the stirred vessel 401 is supplied with water under flow rate control via conduit 421. The ammonium nitrate to be dissolved is fed to the stirred vessel via conduit 420. The feeding is under flow rate control; the flow controller (FC) as follower controller receives its target value from a master controller (QC), which, as quality controller, controls a defined concentration of a component or a defined substitute parameter that permits conclusion of the concentration of a component in the liquid stream drawn off into the primary circuit 402.

The quality controller ensures a constant composition of the solution produced. For example, the conductivity of the solution led off serves as a readily obtainable measure of quality. The level in the stirred vessel 401 is controlled by means of a fill level controller 424 that manipulates the drawing-off of the produced solution 423 through a control valve 430. The process is endothermic, and so the heat removed in the stirred vessel 401 has to be fed back again via the heat transferer 403. For this purpose, heating steam is used, the pressure of which in the feed conduit 404 is controlled by means of a pressure controller 435 that actuates the control valve 409.

The various control circuits are preferably controlled using PID controllers with controller parameters optimized to the desired steady state. The actual value of the heat flow removed in the heat transferer 403 is calculated using the measured value of the temperature Tᵢₙ in the reactor 401, the measured value of the temperature T_{out,1} 412 downstream of the heat transferer 403, the measured value of the mass flow rate \dot{m}₁ in the feed to the heat transferer 303, and the parameter of the heat capacity cₗ for the medium of the liquid flow according to the formula \dot{Q}₁=cₗ\dot{m}₁(Tᵢₙ−T_{out,1}).

As perturbation parameter feedforward, the expected heat flow rate to be fed in on the basis of the ammonium nitrate 420 is registered by means of a heat flow calculator 417 and fed forward as perturbation parameter to the heat flow controller 406.

### EXAMPLES

### COMPARATIVE EXAMPLE 1

The hydroformylation of propene is performed on an industrial scale in a bubble column reactor by means of a rhodium triphenylphosphine complex as catalyst. The reaction releases heat, which has to be removed from the reactor. The chemical engineering process flow diagram for such a process according to the prior art is given in FIG. 1.

The bubble column reactor 101 is supplied with hydrogen and carbon monoxide together as what is called synthesis gas via conduit 120 as gaseous feedstock. Propene is fed in under flow rate control in liquid form as a further feedstock via conduit 121. In order to obtain a liquid phase under the defined reaction conditions, a liquid solvent comprising a catalyst is added under flow rate control via conduit 122. The feedstocks that are gaseous under the reaction conditions are dissolved in the solvent and react in the presence of the homogeneous catalyst to give the desired reaction products. Unconverted gaseous feedstocks, especially hydrogen and carbon monoxide, are removed via the gaseous reactor output 111. The target product of the reaction is removed from the primary circuit 102 via the liquid reactor output 123. Since the reaction is exothermic, the liquid reactor contents are cooled in order to keep the reactor temperature at a desired constant value. This is effected by the cooling of the liquid flow in the primary circuit 102 in the heat transferers 103 and 142, for example with river water as heat transfer medium in the secondary circuits. The various control circuits are controlled using PID controllers with controller parameters optimized to the desired steady state.

### Example 1

The same process as in comparative example 1 is implemented with a controller configuration of the invention as specified in FIG. 2. In this case too, the various control circuits are controlled using PID controllers with controller parameters optimized to the desired steady state. The optimal controller parameters were determined via suitable setting rules as elucidated, for example, in the following specialist literature sources:
Otto Föllinger, Regelungstechnik, 8th edition, 1994, Hüthig Verlag
Heinz Unbehauen, Regelungstechnik I, 13th edition, 2005, Vieweg Verlag
S. Skogestad and Ian Postlethwaite, Multivariable Feedback Control, 2005, Wiley Verlag
The systematic design of PID controllers using Nyquist stability (DOI: 10.23919/ECC.2001.7075961)

In the example shown in FIG. 2, for closed-loop control of the temperature in the reactor 101, a cascade closed-loop controller is provided, comprising a temperature controller 105 as master controller and a heat flow controller 106 as follower controller. A target value for the temperature of the liquid in the reactor 101 is set in the master controller 105. The actual value of the temperature in the reactor 101 is detected by means of a temperature sensor. Depending on the temperature difference between the actual value and the target value of the liquid in the reactor, the master controller 105 calculates, as output signal, a target value for the heat flow removed from or added to the liquid in the primary circuit 102 by the heat transferers 103 and 142. This target value is transmitted to the follower heat flow controller 106, which uses the difference between the actual value and the target value of the heat flow as output signal to generate a control signal for the control valve 108 in the feed to the first heat transferer.

The actual value of the heat flow removed in the first heat transferer 103 is calculated using the measured value of the temperature Tᵢₙ in the reactor 101, the measured value of the temperature T_{out,1} downstream of the first heat transferer 103, the measured value of the mass flow rate \dot{m}₁ through the control valve 108 in the feed to the first heat transferer 103, and the parameter of the heat capacity cₗ for the medium of the liquid flow according to the formula \dot{Q}₁=cₗ\dot{m}₁(Tᵢₙ−T_{out,1}). The actual value of the heat flow removed in the second heat transferer 142 is calculated using the measured value of the temperature Tᵢₙ in the reactor 101, the measured value of the temperature T_{out,2} downstream of the second heat transferer 142, the measured value of the mass flow rate \dot{m}₂ through the control valve 140 in the feed to the second heat transferer 142, and the parameter of the heat capacity cₗ for the medium of the liquid flow according to the formula \dot{Q}₂=cₗ\dot{m}₂(Tᵢₙ−T_{out,2}).

The process according to the prior art and the process of the invention were simulated in the “Aspen Plus Dynamics” simulation tool from AspenTech (20 Crosby Drive, Bedford, MA 01730, U.S.A., www.aspentech.com). The simulation model was based on the following assumptions and parameters:

The total pressure in the reactor 101 is given by the sum total of the partial pressures of hydrogen, carbon monoxide, solvent and propene. No other components in the gas phase are taken into account. The molar proportion of catalyst x_{Cat} remains constant during the simulation. In addition, no heat is exchanged between the reactor 101 or the conduits and the environment, and so adiabatic conditions are assumed. The boundary conditions of the simulation are listed hereinafter:
The heat flow transfer coefficient of the external heat transferers is constant at

$$\\ U = 850 \frac{W}{{m}^{2} K} .$$

The inlet temperature of the heat transfer medium in the secondary circuit is T_{in,C}=48° C.
The outlet temperature of the heat transfer medium in the secondary circuit of the first heat transferer 103 under normal load is T_{out,C,nom}=73° C.
The heat flow withdrawn by means of the first heat transferer 103 under nominal load is \dot{Q}_{HE,nom}=21 600 kW.
The heat flow withdrawn through the second heat transferer 142 in the case of simulation of the perturbation characteristic is \dot{Q}_{HE1,nom}=2160 kW, where the total stationary heat flow through the two heat transferers is \dot{Q}_{TOT,nom}=Q_{Reac,nom}=21 600 kW. In other words, the release of heat remains unaffected by the perturbation.
The liquid flow to the first heat transferer under normal load is

$$\\ {\dot{m}}_{HE , PF} = 1816 \frac{t}{h}$$

The liquid flow to the second heat transferer in the case of simulation of the perturbation characteristic is

$$\\ {\dot{m}}_{HE 1 , PF} = 181.6 \frac{t}{h}$$

The heat flow equation is: \dot{Q}_{HE}=U*A*ΔTₘ=\dot{m}_{PF,HE}c_{PF,l}(T_{in,PF}−T_{out,PF})=\dot{m}_{HE,C}c_{C,l}(T_{out,C}−T_{in,C})
The sole heat sink is the two heat transferers.
The temperature of the liquid flow T_{in,PF} is equal to the reactor temperature T_{R}.
The two heat transferers work in countercurrent: T_{out,PF}=T_{1,PF}, T_{in,PF}=T_{2,PF}, T_{in,C}=T_{1,C} and T_{out,C}=T_{2,C}
The average logarithmic temperature difference is given by:

$$\\ Δ {T}_{m} = \frac{Δ {T}_{1} - Δ {T}_{2}}{ln ( \frac{Δ {T}_{1}}{Δ {T}_{2}} )} = \frac{( {T}_{1 , PF} - {T}_{1 , C} ) - ( {T}_{2 , PF} - {T}_{2 , C} )}{ln ( \frac{( {T}_{1 , PF} - {T}_{1 , C} )}{( {T}_{2 , PF} - {T}_{2 , C} )} )}$$

The reactor temperature under normal load is T_{R}=95° C.
The reaction rate under normal load is

$$\\ {\dot{r}}_{nom} = 5.6 \frac{mol}{lh} .$$

The total pressure in the reactor is p=10 bar.
The partial pressure of carbon monoxide under normal load is p_{CO,nom}=3 bar.
Under normal load, the ratio between hydrogen and carbon monoxide in stream 120 should be chosen such that the partial pressure of hydrogen p_{H}_{₂}_{,nom} is equal to that of carbon monoxide p_{CO,nom}.
The reaction rate is calculated by

$$\\ \dot{r} = {k}_{R , 0} {e}^{{}_{} - \frac{{E}_{{A}_{}}}{{RT}_{R}}} {c}_{Propene} {c}_{Cat} \frac{{c}_{CO}}{1 + {K}_{CO} {c}_{CO}^{{}_{} 2}} \frac{{c}_{{H}_{2}}}{1 + {K}_{{H}_{2}} {c}_{{H}_{2}}}$$

In this equation, c_{CO}, c_{H}_{₂}, c_{Propene} and c_{Cat} are concentrations of the individual components in mol/l, E_{A} is the activation energy, and k_{R,0} is the pre-exponential factor.
The enthalpy of reaction is calculated by the enthalpy model defined in the simulation tool (Aspen Properties) for the liquid phase and the application of Hess's law.

The parameters K_{CO}, K_{H}_{₂}, E_{A} and k_{R,0} of the reaction kinetics were adjusted in order to reflect the negative correlation of the reaction rate with increasing partial carbon monoxide partial pressure. The parameters used are:

$$\\ {K}_{CO} = 45000 \frac{{dm}^{6}}{{mol}^{{}_{} 2}} , {K}_{{H}_{2}} = 500 \frac{{dm}^{3}}{mol} , {E}_{A} = 60000 \frac{J}{mol} and$$

$$\\ {k}_{R , 0} {c}_{Cat} = 4.2465 {10}^{15} \frac{{mol}^{{}_{} 2}}{{dm}^{{}_{} 3} h} .$$

Since the catalyst concentration c_{Cat} is unknown but constant, this was combined with the preexponential factor k_{R,0}. This is equivalent to the numerical value of 1 for the catalyst concentration c_{Cat}. The reaction rate is a function of the concentrations neglecting the partial pressures.

The correlation between the partial pressures and the concentrations can be ascertained using the known solubilities of the individual gases in the liquid phase. The solubility of the gases depends on the solvent in which the reaction proceeds. The solvent is generally a mixture of rhodium catalyst, ligand and n-butyraldehyde, isobutyraldehyde, dimers, trimers and higher oligomers of butyraldehyde. This means that the solvent consists mainly of high-boiling components that are formed by the side reactions of the hydroformylation. By way of simplification, it was assumed that the mixture (including catalyst and ligand) can be described by the simulation of a single representative component. The Texanol component was selected as solvent since its parameters are available in Aspen Properties and these are in good agreement with the real mixture described.

The physical state equation “Predictive Soave Redlich Kwong” (PSRK) was used in order to ascertain the solubility and other properties and to conduct the dynamic simulations. Solubility is a function of pressure, temperature and the components of the liquid phase, which is calculated by the state equation.

In order to set the dimensions of the reactor 101, it is necessary to define a conversion for a single pass of synthesis gas 120 and propene 121. The conversion in a single pass X_{SP,Propene} is defined here as the quotient of the flow rate of butanal \dot{n}_{Butanal,Rework} which is fed to the workup section to the flow rate of propene \dot{n}_{Propene,Feed} which is fed to the reactor 101. This definition was chosen since a considerable portion of unconverted propene is present in the offgas and hence the sum would have to be formed over several streams if the unconverted propene had to be ascertained. The conversion for a single pass of the synthesis gas 120 is adjusted such that the molar proportion of carbon monoxide in the gaseous reaction output y_{CO,Vent,nom} in the workup direction is

$$\\ 0.3 \frac{mol}{mol} .$$

The synthesis gas composition in the feed stream is adjusted such that the molar proportion of hydrogen in the gaseous reaction output under normal load is equal to the molar proportion of carbon monoxide in the gaseous reaction output under nominal load. The partial pressure of hydrogen and carbon monoxide is about 3 bar, and the hydroformylation reaction is in the region of negative correlation between the partial pressure of carbon monoxide and the reaction rate.

A comparison of the simulation results between comparative example 1 and example 1 for the control characteristics of the controller in the event of a change in the target value of the reactor temperature from 95° C. to 92° C. and back to 95° C. is shown in FIG. 6 and FIG. 7. In this case, no second heat transferer 142 is added for either example.

In comparative example 1, a temperature-temperature cascade is used in order to control the temperature in the reactor. By contrast, in inventive example 1, a temperature-heat flow cascade is used in order to control the temperature in the reactor. In order to achieve equilibration of the method in the simulations, in both cases, the boundary conditions of the method are kept constant for the first 60 minutes, for example the reactor temperature or the reactant streams. This is done using “Aspen Plus Dynamics”. After one hour of simulated time, the target value of the reactor temperature is lowered instantaneously from 95° C. to 92° C. After a further four hours of simulated time, the target value of the reactor temperature is reset to its original value of 95° C. In FIG. 6 and FIG. 7, the dotted line in each case shows the target value of the reactor temperature, the solid line the actual value of the reactor temperature, and the dashed-and-dotted line the control signal of the master controller 105. In the case of the comparative example this is a target temperature for the follower controller, and in the case of the inventive example a target heat flow for the follower controller. As is clearly apparent from a comparison of the curves for the actual values of the reactor temperature, the method of the invention reaches the target value significantly more quickly than the method according to the prior art. The control quality is thus better.

For comparative example 1 and inventive example 1, simulation calculations are conducted for a further scenario. After a simulation time of one hour, the propene mass flow rate 121 to the reactor is instantaneously lowered by 10%. After a further four hours of simulation time, the propene mass flow rate is reset to its original value. In this case too, no second heat transferer 142 is added for either example.

In FIG. 8 and FIG. 9, the dotted line in each case shows the target value of the reactor temperature, the solid line the actual value of the reactor temperature, and the dashed-and-dotted line the control signal of the master controller 105. As in the case of the jump in target value, it is clearly apparent from a comparison of the curves for the actual values of the reactor temperature that the method of the invention reaches the target value significantly more quickly than the method according to the prior art. The control quality is thus better in this scenario too.

For comparative example 1 and inventive example 1, the simulation calculations that follow are conducted for a further scenario. After one hour of simulation time, a second heat transferer 142 is added. The heat flow which is removed from the liquid in the primary circuit by the second heat transferer was fixed at 10% of the heat flow removed up to that point by the first heat transferer 103. The amount of heat released by the reaction remains unaffected. After five hours of simulation time, the second heat transferer 142 is shut down again, and the first heat transferer again assumes the entire heat flow.

In FIG. 10 and FIG. 11, the dotted line in each case shows the target value of the reactor temperature, the solid line the actual value of the reactor temperature, and the dashed-and-dotted line the control signal of the master controller 105. As in the case of the previous simulation examples, it is clearly apparent from a comparison of the curves for the actual values of the reactor temperature that the method of the invention reaches the target value significantly more quickly than the method according to the prior art. The control quality is thus better in this scenario too.

These simulation results show that, when a temperature-heat flow cascade is used, much better control quality is achieved than when a temperature-temperature cascade according to the prior art is used.

1.-12. (canceled)

13. A method of closed-loop control of the temperature in a chemical engineering apparatus, in which, in a primary circuit, a liquid is conveyed out of the apparatus, fed at least partly to a heat transferer and recycled at least partly back to the apparatus, where the heat transferer is cooled or heated by a heat transfer medium in a secondary circuit, comprising the steps of
providing a target value for the temperature of the liquid in the apparatus,
detecting an actual value for the temperature of the liquid in the apparatus,
calculating the temperature difference between the actual value and the target value of the liquid in the apparatus,
wherein
a heat flow taken from or added to the liquid in the primary circuit by the heat transferer is ascertained,
a control signal is calculated on the basis of a defined closed-loop control algorithm, where the closed-loop control algorithm is configured such that the control signal is dependent on the heat flow and the temperature difference between the actual value and the target value of the liquid in the apparatus, and
the flow rate of the stream of liquid through the heat transferer in the primary circuit and/or a flow rate of the heat transfer medium through the heat transferer in the secondary circuit is/are manipulated on the basis of the control signal.

14. The method according to claim 13, wherein the heat flow is ascertained from the liquid flow through the heat transferer in the primary circuit and from a temperature difference between the temperature of the liquid upstream and downstream of the heat transferer in the primary circuit and/or the heat flow is ascertained from a flow rate of the heat transfer medium through the heat transferer in the secondary circuit and from a temperature difference between the temperature of the heat transfer medium upstream and downstream of the heat transferer in the secondary circuit.

15. The method according to claim 13, wherein the closed-loop control algorithm comprises a temperature controller and a heat flow controller, where the output signal calculated by the temperature controller is dependent on the temperature difference between the actual value and the target value of the liquid in the apparatus and is a target value for the heat flow taken from or added to the liquid in the primary circuit through the heat transferer, this target value is transmitted to the heat flow controller, and the output signal generated by the heat flow controller is dependent on the difference between the actual value and the target value of the heat flow and is the control signal for manipulation of the liquid flow through the heat transferer in the primary circuit and/or for manipulation of the flow rate of the heat transfer medium through the heat transferer in the secondary circuit.

16. The method according to claim 13, characterized in that there is at least one bypass in the primary circuit that runs parallel to the heat transferer.

17. The method according to claim 13, characterized in that there is at least one control unit for manipulation of the liquid flow on the basis of the control signal in the primary circuit in the feed to the heat transferer and/or in the drain from the heat transferer and/or in the bypass.

18. The method according to claim 13, characterized in that there is at least one control unit for manipulation of the flow rate of the heat transfer medium on the basis of the control signal in the secondary circuit in the feed to the heat transferer and/or in the drain from the heat transferer.

19. The method according to claim 13, wherein the closed-loop control algorithm has a split range control function where, rather than one control signal, at least two control signals are calculated and at least two streams in the primary circuit and/or in the secondary circuit are manipulated, where a first stream is manipulated as a function of a first control signal and a second stream as a function of a second control signal.

20. The method according to claim 13, characterized in that the liquid flow conveyed from the apparatus into the primary circuit is set to a defined target value.

21. The method according to claim 13, characterized in that the closed-loop control algorithm, in calculating the control signal, takes account of at least one perturbation parameter, where the perturbation parameter comprises a measured or estimated process parameter, especially at least one raw material flow and/or at least one further heat flow.

22. The method according to claim 13, characterized in that a chemical or biological, endothermic or exothermic reaction or an exothermic or endothermic physical process is proceeding in the apparatus.

23. The method according to claim 22, wherein the reaction is a hydroformylation, etherification, ether cleavage, enalization, dehydrogenation, pyrolysis, hydrogenation, or a cracking process.

24. The method according to claim 23, wherein the hydrogenation is a full hydrogenation, selective hydrogenation or ring hydrogenation.
